(19) United States
(12) Patent Application Publication
(10) Pub. No.: US 2015/0289918 A1
(43) Pub. Date: Oct. 15, 2015

(54) Sterile-Packaged Disposable Contouring Tool Systems for Medical Implants and Methods for Contouring Medical Implants

(71) Applicant: Flower Orthopedic Corporation, Philadelphia,, PA (US)

(72) Inventors: Oliver Burckhardt, Philadelphia, PA (US); Josef Zrinski, Wurmlingen (DE); Davor Zrinski, Rietheim-Weilheim (DE)

(21) Appl. No.: 14/442,045

(22) PCT Filed: Nov. 8, 2013
(86) PCT No.: PCT/US2013/069195

§ 371 Date: May 11, 2015

Related U.S. Application Data
(60) Provisional application No. 61/725,139, filed on Nov. 12, 2012.

(51) Int. Cl. A61B 17/88

(52) U.S. Cl. CPC A61B 17/8863

(57) ABSTRACT

Systems and methods contour a medical implant and provide a first tool, a second tool and a medical implant, wherein each of the first tool, the second tool and the medical implant are individually sterilized and packaged. After unpackaging the sterile first tool, the sterile second tool and the sterile medical implant, a first fixed portion of the sterile medical implant is inserted into, and held by, a first channel of the first tool. A second fixed portion of the sterile medical implant is inserted into, and held by, a second channel of the second tool. The sterile first tool is moved relative to the sterile second tool to apply deformation forces to the at least a portion of the sterile medical implant for contouring the sterile medical implant at a deformation portion of the sterile medical implant, located between the sterile first and second tools.

### FIELD OF THE DISCLOSURE

This application claims the benefit of U.S. Provisional Patent Application No. 61/725,139, filed Nov. 12, 2012, the entirety of which is hereby incorporated by reference into this application.

The present disclosure relates to sterile-packaged disposable contouring tool systems for medical implants and methods for contouring medical implants. The medical implants may be contoured, shaped and/or manipulated with the contouring tool systems and methods prior to affixing the medical implants.

### BRIEF DESCRIPTION OF THE DRAWING

So that the features and advantages of the present disclosure can be understood in detail, a more particular description of the systems and methods, may be had by reference to the embodiments thereof that are illustrated in the appended drawings. It is to be noted, however, that the appended drawings illustrate only some typical embodiments of the present systems and methods and are therefore not to be considered limiting of its scope, for the systems and methods may admit to other equally effective embodiments.

FIG. 1 illustrates a perspective view of a contouring tool in an embodiment.

FIG. 2 illustrates a perspective view of a contouring tool in an embodiment.

FIG. 3 illustrates a perspective view of a contouring tool system in an embodiment.

FIG. 4 illustrates a perspective view of a contouring tool system in an embodiment.

FIG. 5 illustrates a perspective view of a contouring tool system in an embodiment.

FIG. 6 illustrates a perspective view of a contouring tool system in an embodiment.

### DETAILED DESCRIPTION OF THE DISCLOSURE

The present contouring tool systems and methods may contour, shape and/or manipulate one or more medical implants prior to affixing the medical implants to bones. Medical implants frequently require bending, twisting, contouring and/or shaping so that the medical implants may have a contour and/or shape that corresponds to a contour and/or shape of a bone before being affixed to the bone. The present systems and/or methods may provide inexpensive, sterile-packaged, disposable contouring tools for easily bending, twisting, manipulating, contouring and/or shaping one or more medical implants. It should be understood that contouring the medical implant may also mean angling the implant, bending the implant, twisting the implant, turning the implant, manipulating the implant, and/or shaping the implant.

The present disclosure relates to sterile-packaged disposable contouring tool systems for medical implants and/or methods for contouring one or more medical implants. The one or more medical implants may be sterile-packaged medical implants which may be contoured with the present tool systems and/or methods prior to affixing the one or more medical implants to at least one bone. In embodiments, the present tool systems and/or medical implants may be, for example, sterile-packaged, single-use products and/or disposable products.

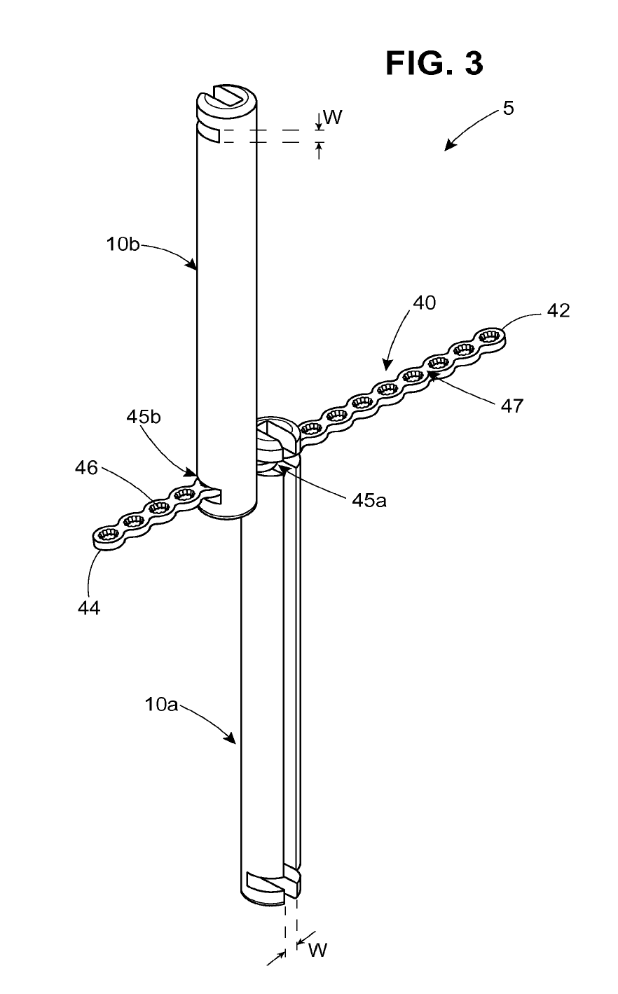

Referring now to the drawings wherein like numerals refer to like parts, the medical implants may be, for example, at least one sterile-packaged bone-fixation implant or bone plate 40 (hereinafter “plate 40”), as shown in FIG. 3, which may be attached and/or connected to at least one bone. The plate 40 may be attached and/or connected to at least one bone via, for example, one or more locking and/or non-locking screws. The plate 40 has a length defined between a first end 42 and a second end 44 which is located opposite to the first end 42 of the plate 40. The plate 40 has one or more holes 46 formed therein and separated by one or more partitions 47. Each of the one or more holes 46 of the plate 40 are sized, shaped and/or configured to receive a locking or non-locking screw.

In embodiments, the plate 40 may be, for example, an anatomic plate, a reconstruction plate or an osteosynthesis plate. In an embodiment, the plate 40 may be, for example, a veterinary orthopedic implant. Outer surfaces of the plate 40 may be made of one or more biomedical materials, such as, for example, titanium, silicone, apatite and/or the like. In embodiments, the plate 40 may be, for example, a hand plate, a foot plate, a reconstruction S-plate, a reconstruction M-plate, a proximal humerus plate, a distal radius plate or an osteosynthesis S-plate. In an embodiment, the hand and/or foot plate may be, for example, a straight plate, a L-plate, an oblique T-plate, a T-plate, a H-plate, a mediocarpal plate, an elongated L-plate, a straight plate with slots, or an angled and/or inclined plate. In an embodiment, the reconstruction S-plate may be, for example, a straight plate or a T-plate. In an embodiment, the distal radius plate may be, for example, volar, narrow L-plate, a volar, narrow R-plate, a volar, wide L-plate, a volar, wide R-plate, a dorsal L-plate, a dorsal R-plate, a volar L-plate or a volar R-plate. It should be understood that the present disclosure is not limited to a specific embodiment of the plate 40 and/or the one or more biomedical materials on the outer surfaces of the plate 40. Moreover, the plate 4 may be any bone plate or bone-fixation implant as known to one of ordinary skill in the art.

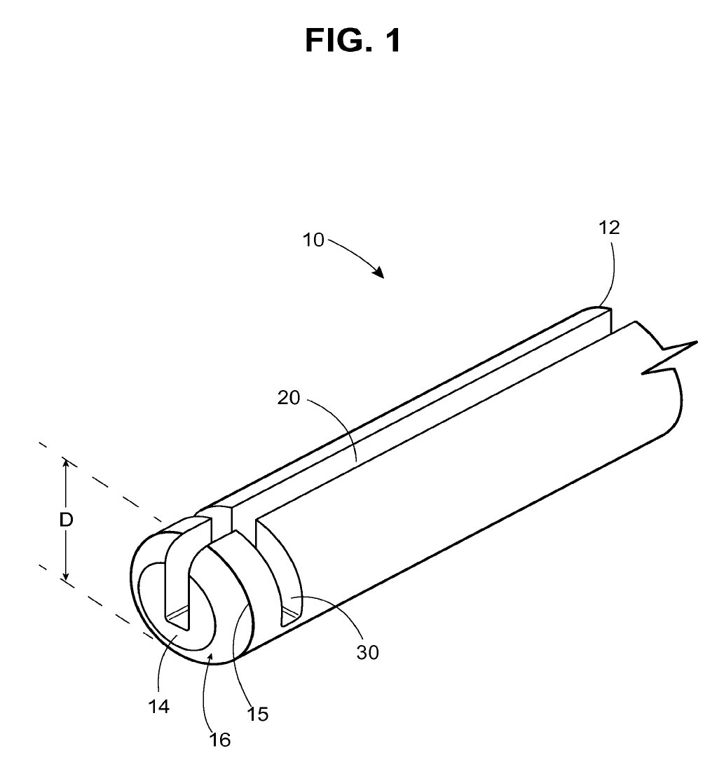

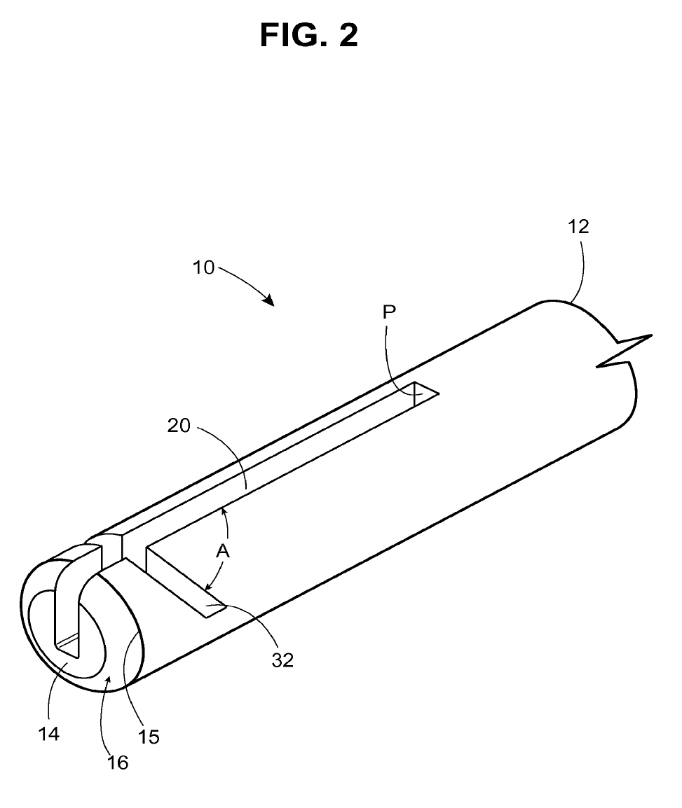

As shown in FIGS. 1 and 2, a individually sterile-packaged, disposable contouring tool 10 (hereinafter “tool 10”) of the present disclosure has a length defined between a first end 12 and a second end 14 that is located opposite to the first end 12 of the tool 10. In embodiments, the tool 10 may have, for example, a rod-like shape, a cylindrical shape or a rectangular shape. The tool 10 may have an outer perimeter or circumference 15 (hereinafter “circumference 15”) wherein one or more channels may be formed therein. For example, the tool 10 may have a longitudinal channel 20 extending at least a portion of the length of the tool 10. For example, the longitudinal channel 20 may have a length that is equal to or substantially equal to the length of the tool 10 as shown in FIGS. 1, 3 and 5. In an embodiment, the longitudinal channel 20 may have a length that is less than the length of the tool 10. For example, the longitudinal channel 20 may extend from the second end 14 to a point P which is located between the second end 14 and the first end 12 of the tool 10 as shown in FIG. 2. As a result, the first end 12 of the tool 10 may be solid non-channeled shape, without the longitudinal channel 20 extending therethrough.

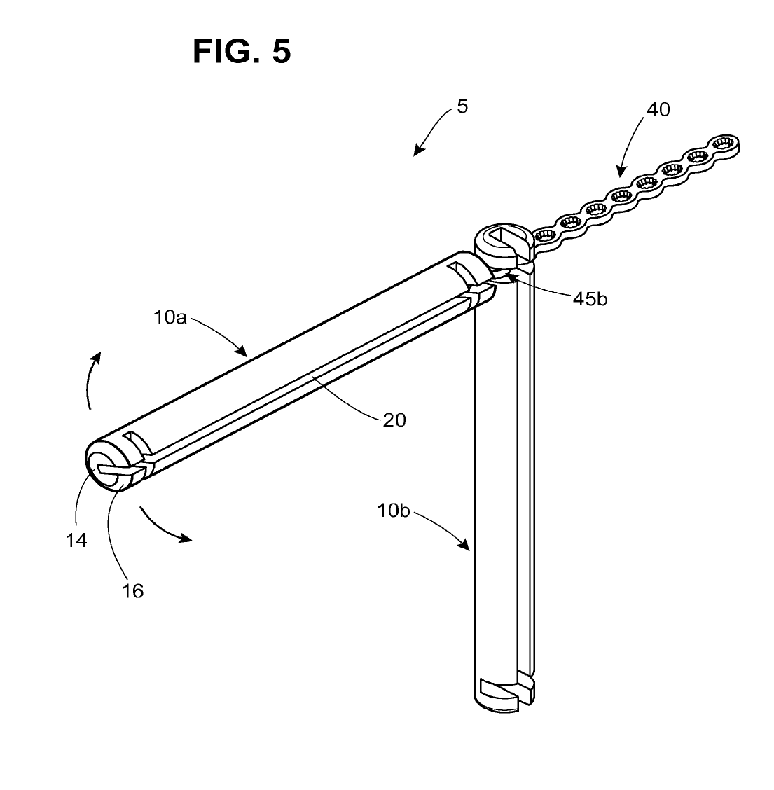

The tool 10 may have at least one lateral channel 30, as shown in FIG. 1, and/or at least one angled channel 32, as shown in FIG. 2, which may be located adjacent to the first end 12 and/or the second end 14 of the tool 10. In embodiments, the tool 10 may have lateral channels 30 and/or angled channels 32 located adjacent to the first end 12 and the second end 14 as shown in FIGS. 3 and 5. In an embodiment, at least one lateral channel 30 may be perpendicular or substantially perpendicular with respect to the length of the longitudinal channel 20 as shown in FIG. 1. In embodiments, at least one angled channel 32 may formed at an angle A with respect to the length of the tool 10 as shown in FIG. 2. The angle A of the at least one angled channel 32 is less than 90 degrees, such as, for example, about 30 degrees, about 45 degrees or about 60 degrees.

The longitudinal channel 20, the lateral channel 30 and/or the angled channel 32 (collectively referred to hereinafter as “channels 20, 30, 32”) may extend or be formed inwardly from the outer circumference 15 of the tool 10 to a depth D, as shown in FIG. 1. In an embodiment, the depth D may be greater than or equal to half of the diameter of the tool 10. In an embodiment, the depths D for the channels 20, 30, 32 may be the same or substantially the same; however, in other embodiments, the depths D for each the channels 20, 30, 32 may be different depths. The channels 20, 30, 32 are sized, shaped and/or configured to receive at least a portion of the plate 40. For example, the depth D for the channels 20, 30, 32 may be sized, shaped and/or configured to receive at least a portion of the plate 40. As a result, at least a portion of the plate 40 may be inserted into and/or positioned within the channels 20, 30, 32 of the tool 10 for contouring the plate 40.

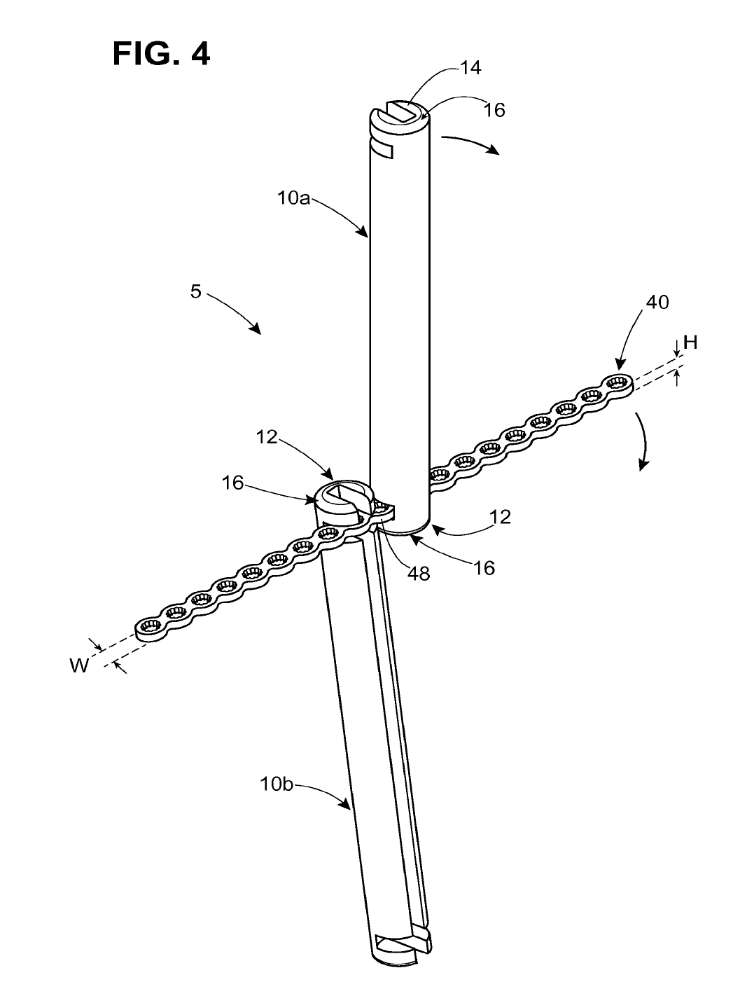

In embodiments, the depth D of the channels 20, 30, 32 of the tool 10 may be greater than or equal to an overall width w of the plate 40, shown in FIG. 4. As a result, the overall width w of the plate 40 along a portion of the length of the plate 40 may be inserted into and/or positioned within the depth D of the channels 20, 30, 32 of the tool 10 for contouring the plate 40. The portion(s) of the length of the plate 40 inserted into and/or positioned within depth D of the channels 20, 30, 32 for contouring the plate 40 is referred to hereinafter as a first fixed portion 45a and/or a second fixed portion 45b (collectively known hereinafter as “fixed portions 45a, 45b”) as shown in FIGS. 3-5.

The channels 20, 30, 32 of the tool 10 may have a width W that is sized, shaped and/or configured to firmly and securely receive and hold the tool 10, having a height H, that is inserted into and/or positioned within one of the channels 20, 30, 32. In embodiments, the width W of the channels 20, 30, 32 are equal to, substantially equal to or greater than the height H of the plate 40. As a result, when, for example, the first fixed portion 45a of the plate 40 is inserted into or positioned within one of the channels 20, 30, 32 having the specifically sized or configured depth D and width W, the first fixed portion 45a of the plate 40 is firmly and securely held, received and/or maintained within the tool 10 for contouring the plate 40. As a result, the plate 40 may be firmly and securely connected and/or attached to the tool 10 via the first fixed portion 45a of the plate 40 and one of the channels 20, 30, 32 formed in the tool 10. By firmly and securely connecting, attaching or holding the first fixed portion 45a of the plate 40 within one of the channels 20, 30, 32 of the tool 10, remaining portions of the plate 40 outside of the tool 10 may be pushed, pulled, moved, twisted and/or bent to contour the plate 40.

In embodiments, a contouring guide surface 16 (hereinafter “surface 16”) may extend from the outer circumference 15 of the tool 10 to the second end 14 of the tool 10 (as shown in FIGS. 1 and 2) and/or to the first end 12 of the tool 10 (as shown in FIGS. 3-5). At least a portion of the surface 16 of the tool 10 may have a cross-sectional profile which may extend from the circumference 15 of the tool 10 to the first end 12 or the second end 14 of the tool 10. The cross-section profile of the surface 16 may be linear, flared, angled, rounded and/or curved at one or more locations between the outer circumference 15 and the first end 12 or the second end 14 of the tool 10. For example, the cross-sectional profile of the surface 16 may have a concaved portion, a convex portion, a linear portion or combinations thereof. In an embodiment, the cross-sectional profile of the surface 16 may be, for example, a convex surface, as shown in FIGS. 1 and 2, for contouring or bending the plate 40.

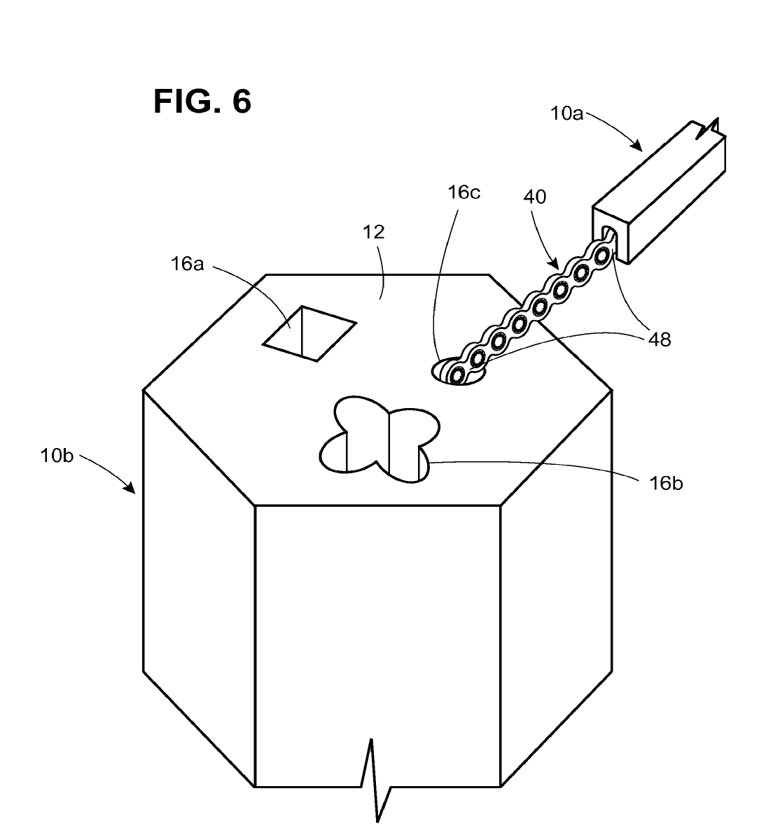

FIGS. 3-5 show a sterile-packaged, disposable contouring tool system 5 which may comprise at least a first contouring tool 10a (hereinafter “first tool 10a”) and a second contouring tool 10b (hereinafter “second tool 10b”). In embodiments, the first tool 10a and/or the second tool 10b (hereinafter “first and second tools 10a, 10b”) are the same as or substantially the same as the tool 10 having the same channels 20, 30, 32 with the same depth D and the same width W for receiving and holding fixed portions 45 of the plate 40 within the first and second tools 10a, 10b for contouring the plate 4. The first and second tools 10a, 10b may have the same surface 16 which may extend from the same circumference 15 to the first end 12 or the second end 14 of the first and second tools 10a, 10b. In embodiments, the first and second tools 10a, 10b may have, for example, a rod-like shape, a cylindrical shape or a rectangular shape. In embodiments, the first and second tools 12, 14 may be the same or substantially the same size having the same or substantially the same dimensions. In an embodiment, the first and second tools 12, 14 may have different sizes with different dimensions as shown in FIG. 6.

In embodiments, cross-sectional shapes of the first and second tools 10a, 10b may be the same or substantially the same as shown in FIGS. 1-5 or may be different as shown in FIG. 6. For example, the cross-sectional shapes of the first and second tools 10a, 10b may be, for example, circles, as shown in FIGS. 1-5, or ovals (not shown in the drawings). However, in other embodiments, the outer cross-sectional shapes of the first and second tools 10a, 10b may be polygons, such as, for example, triangles, squares, hexagons, as shown in FIG. 6. In an embodiment, the first and second tools 10a, 10b may have an ergonomic design (not shown in the drawings) whereby the cross-sectional shapes of the first and second tools 10a, 10b may comprise one or more different shapes along the length of the first and second tools 10a, 10b such that the first and second tools 10a, 10b may comfortably fit into, or be held by, hands of a user.

After the first fixed portion 45a and a second fixed portion 45b of the plate 40 are firmly and securely held within the channels 20, 30, 32 of the first and second tools 10a, 10b, the first and second tools 10a, 10b are used in cooperation with one another to contour the plate 40. Orientations of the channels 20, 30, 32 of the first and second tools 10a, 10b are configured such that one or more portions of the plate 40 may be manipulated and/or contoured in different directions when the plate 40 is firmly and securely connected to, attached to or held within the first and second tools 10a, 10b. Moreover, the orientations of the channels 20, 30, 32 of the first and second tools 10a, 10b may be configured such that different sized bone plates (not shown in the drawings) may be manipulated and/or contoured in different directions by using the first and second tools 10a, 10b in cooperation with one another.

FIG. 3 illustrates an embodiment of the first and second tools 10a, 10b being utilized in cooperation with one another to contour the plate 40. The plate 40 may be firmly and securely inserted into, for example, the lateral channels 30 of the first and second tools 10a, 10b. As a result, the plate 40 is firmly and securely connected to, attached to or held within the lateral channels 30 of the first and second tools 10a, 10b. For example, the first fixed portion 45a, located adjacent to the first end 42 of the plate 40, may be firmly and securely inserted into the lateral channel 30 of the first tool 10a, and a second fixed portion 45b, located adjacent to the second end 44 of the plate 40, and may be firmly and securely inserted into the lateral channel 30 of the second tool 10b as shown in FIG. 3. As a result, the fixed portions 45a, 45b are firmly and securely held within the lateral channels 30 for contouring the plate 40. Moreover, the plate 40 is firmly and securely connected or attached to the first and second tools 10a, 10b via the lateral channels 30 and the fixed portions 45a, 45b of the tool 40.

A user then holds the first and second tools 10a, 10b, one in each hand, and moves the first and second tools 10a, 10b relative with respect to one another to contour the plate 40 by, for example, pushing, pulling, bending and/or twisting the plate 40 via the first and second tools 10a, 10b. While the first and second tools 10a, 10b are being moved with respect to one another, the fixed portions 45a, 45b firmly and securely held within the first and second tools 10a, 10b, respectively, are maintain by, for example, frictional forces such that the plate 40 may be contoured between the first and second tools 10a, 10b. By moving the first and second tools 10a, 10b with respect to one another, the user applies deformation forces to a deformable portion 48 of the plate 40 that is located between the fixed portions 45a, 45b and between the first and second tools 10a, 10b as shown in FIG. 4. The deformable portion 48 of the plate 40 includes at least a portion of the length of the plate 40 between the first end 42 and the second end 44 of the plate 40. For example, the deformable portion 48 of the plate 40 may comprise one or more holes 46 and/or one or more partitions 47 extending along the length of the plate 40 between the first end 42 and the second end 44.

The deformation forces may be applied by the user by the user pushing, pulling, bending, twisting and/or leveraging the first and second tools 10a, 10b with respect to one another. As a result, the fixed portions 45a, 45b firmly and securely connected to, attached to and/or held within the first and second tools 10a, 10b, respectively, move with respect to each other which, in turn, causes or creates a deformation of the plate 40 at the deformation portion 48 of the plate 40 located between the fixed portions 45a, 45b and between the first and second tools 10a, 10b. For example, the user may twist the first and second tools 10a, 10b towards one another which applies twisting deformation forces to the fixed portions 45a, 45b which, in turn, twists the plate 40 at the deformation portion 48 between the first and second tools 10a, 10b.

Additional contouring of the plate 40 may be achieved by positioning different portions of the plate 40 within the channels 20, 30, 32 of the first and second tools 10a, 10b and subsequently applying deformation forces to a different deformation portion of the plate 40 located between the first and second tools 10a, 10b. For example, the plate 40 may be removed from the first and second tools 10a, 10b, and different portions of the plate 40 may be inserted into one of the channels 20, 30, 32 of the first and second tools 10a, 10b to serve as or to be utilized as new fixed portions 45a, 45b. A different portion of the plate 40 may be inserted into lateral channel 30 of the first tool 10a to be utilized as a new first fixed portion 45a, and another different portion of the plate 40 may be inserted into lateral channel 30 of the second tool 10b to be utilized as a new second fixed portion 45b. The user may apply deformation forces to the new fixed portions 45a, 45b by moving the first and second tools 10a, 10b relative to one another for contouring a different portion of the plate 40. As a result, a deformation of the plate 40 at a new deformation portion 48, located between new fixed portions 45a, 45b and the first and second tools 10a, 10b, is caused or created by deformation forces applied to the plate 40 by moving the first and second tools 10a, 10b relative to one another. Additional deformations of the plate 40 may be created by applying additional deformation forces along one or more different portions of the length of the plate 40. As a result, one or more different portions of the length, or the entire length, of the plate 40 may be contoured or shaped to correspond to a contour or shape of the bone subsequently receiving the plate 40.

FIG. 4 illustrates an embodiment of contouring tool system 5 having the fixed portions 45a, 45b of the plate 40 firmly and securely inserted within the lateral channels 30 of the first and second tools 10a, 10b, respectively. As a result, the plate 40 is firmly and securely connected or attached to or held within the first and second tools 10a, 10b. The first fixed portion 45a of the plate 40 may be located adjacent to the first end 42 of the plate 40, and the second fixed portion 45b of the plate 40 may be located adjacent to the second end 44 of the plate 40. The first fixed portion 45a of the plate 40 may connect and/or attach the plate 40 to the first tool 10a via the lateral channel 30 of the first tool 10a. As a result, the first tool 10a may extend, for example, upwardly with respect to a top side from the plate 40. The fixed portion 45b of the plate 40 may connect and/or attach the plate 40 to the second tool 10b via the lateral channel 30 of the second tool 10b. As a result, the second tool 10b may extend, for example, downwardly with respect to a bottom side of the plate 40. Moreover, the lengths of the first and second tools 10a, 10b may extend away from one another and/or may be parallel or substantially parallel with respect to one another.

In embodiments, the user, with each hand holding one of the first and second tools 10a, 10b, may, for example, move or pull the first tool 10a downward towards the plate 4 (as shown by curved downward arrow near the top of first tool 10a in FIG. 4) or may move or push the first tool 10a downward towards the first end 12 of the second tool 10b (not shown in the drawings). By moving or pulling the first tool 10a downward towards the plate 40, the user applies bending deformation forces to the fixed portions 45a, 45b firmly and securely held within the first and second tools 10a, 10b, respectively. As a result, the deformation portion 48 of the plate 40 located between the first and second tools 10a, 10b is deformed or bent downwardly by the bending deformation forces applied to the plate 40 by the user and/or the first and second tools 10a, 10b (as shown by curved downward arrow located near the right end of plate 40 in FIG. 4. Moreover, the plate 40 is contoured or bent downwardly at the deformation portion 48 of the plate 40 between the first end 42 and the second end 44 of the plate 44. By moving or pushing the first tool 10a towards the first end 12 of the second tool 10b, the user applies bending deformation forces to the fixed portions 45a, 45b which upwardly deforms or bends the deformation portion 48 of the plate 40. As a result, that the plate 40 is contoured or bent upwardly at the deformation portion 48 of the plate 40 between the first end 42 and the second end 44 of the plate 40.

In embodiments, the first and second tools 10a, 10b may have the surface 16 located adjacent to the first end 12 and/or second end 14 of the first and second tools 10a, 10b as shown in FIG. 4. The cross-sectional profile of the surface 16 of the first and second tools 10a, 10b may provide or serve as a fulcrum for a lever formed by at least one of the first and second tools 10a, 10b and the plate 40. The fulcrum, provided by the cross-section profile of the surface 16, is a pivot point for the lever formed by at least one of the first and second tools 10a, 10b and the plate 40 when the first and second tools 10a, 10b are pivoted onto one another to contour the plate 40. For example, cross-sectional profile of the surface 16 located at the first end 12 of the first tool 10a provides a fulcrum between the first and second tools 10a, 10b when the first tool 10a is moved or pulled downward with respect to the top side of the plate 40 as shown in FIG. 4. Further, the fulcrum provided by the surface 16 is a pivot point for the lever formed by the first tool 10a and the plate 40 which applies downward bending deformation forces to the first fixed portion 45a held within the lateral channel 30 of the first tool 10a. As a result, the plate 40 is bent downward at the deformation portion 48 by the downward bending deformation forces.

On the other hand, the surface 16 located at the first end 12 of the second tool 10b provide a fulcrum between the first and second tools 10a, 10b when the first tool 10a is moved or pushed downward with respect to the first end 12 of the second tool 10b as shown in FIG. 4. The fulcrum provided by the surface 16 of the second tool 10a is a pivot point for the lever formed by the first tool 10a and the plate 40 which applies upward bending deformation forces to the first fixed portion 45a held within the lateral channel 30 of the first tool 10a. As a result, the plate 40 is bent upward at the deformation portion 48 by the upward bending deformation forces. With the surface 16 of the first and second tools 10a, 10b providing a fulcrum for levers from by the first and second tools 10a, 10b and plate 40, leverage may be applied to the first and second tools 10a, 10b when the user moves the first and second tools 10a, 10b relative to one another. As a result, the contouring to tool system provides a mechanical advantage to the user when pivoting the first and second tools 10a, 10b on the fulcrum provided by the surface 16 for applying bending deformation forces to the plate 40. Moreover, the principle mechanism for bending the plate 40 is achieved by the length of the first and second tools 10a, 10b such that greater bending movement and/or forces may applied to the plate 40 by utilizing first and second tools 10a, 10b having longer lengths and applying lesser amount of force to the first and second tools 10a, 10b having longer lengths.

FIG. 5 shows the contouring tool system 5 wherein the first fixed portion 45a of the plate 40 is firmly and securely inserted into and held within the longitudinal channel 20 of the first tool 10a. As a result, the plate 40 is firmly and securely connected or attached to the first tool 10a via the first fixed portion 45a and the longitudinal channel 20 of the first tool 10a. The second fixed portion 45b of the plate 40 is firmly and securely inserted into and held within the lateral channel 30 of the second tool 10b. As a result, the plate 40 is firmly and securely connected or attached to the second tool 10b via the second fixed portion 45b and the lateral channel 30 of the second tool 10b.

In an embodiment, the user may move, push or pull the first tool 10a upwardly or downwardly with respect to the plate 40 to apply upward or downward deformation forces to the plate 40 (as shown by the curved arrows located near the second end 14 of the first tool 10a in FIG. 5. The surface 16 of the first tool 10a serves the fulcrum between the first and second tools 10a, 10b for applying upward or downward bending deformation forces to the first fixed portion 45a of the plate 40 firmly and securely held within the first tool 10a. As a result, the deformation portion 48 of the plate 40, located between the first and second tools 10a, 10b may be deformed or bent upwardly or downwardly by the upwardly or downwardly bending deformation forces applied to the first fixed portion 45a of the plate 40 by the first tool 10a.

FIG. 6 illustrates a first tool 10a which may have a first end 42 of the plate 40 firmly and securely inserted within a longitudinal channel 20 of the first tool 10a. In an embodiment, the cross-section shape of the first tool 10a is, for example, a polygon,. One of the first end 12 (as shown in FIG. 6) and/or the second end 14 of the second tool 10b may have one or more bores 16a, 16b, 16c formed therein. The one or more bores formed in the first end 12 and/or the second end 14 of the second tool 10b may have one or more cross-section shapes, such as, for example, a polygon cross-section shapes (see first bore 16a), a non-polygon cross-section shapes (see second bore 16b), circular or rounded cross-section shapes (see third bore 16c), or combinations thereof. The one or more bores 16a, 16b, 16c may be sized, shaped and/or configured to firmly and securely receive and/or hold at least a portion of the plate 40 which may extend outwardly with respect to the first tool 10a which may be referred to as the first fixed portion 45a of the plate 40. The first fixed portion 45s of the plate may be inserted into one of the bores 16a, 16b, 16c, as shown in FIG. 6. As a result, the plate 40 may be firmly and securely connected and/or attached to the first tool 10a via the longitudinal channel 20 of the first tool 10a and/or may connect the first tool 10a to the second tool 10b via one of the bores 16a, 16b, 16c, as shown in FIG. 6.

The user may move, rotate, bend and/or twist the first and second tools 10a, 10b with respect to one another to apply deformation forces to a deformation portion 48 of the plate 40 which is located adjacent to the first end 12 of the second tool 10b. The deformation forces applied to the first and second tools 10a, 10b by the user may deform, curve, twisting, move the deformation portion 48 with respect to the fixed portion 45 of the plate 40 that is firmly and securely connect and/or attached to the second tool 10b. As a result, one or more deformations, bends and/or twists may be form at or adjacent to the deformation portion 48 of the plate 40 for deforming and/or contouring the plate 40 with the first and second tools 10a, 10b.

FIG. 6 also shows the first end 12 or first fixed portion 45a of the second tool 10b which is a solid end or has a solid portion, meaning there are no channels 20, 30, 32 formed within the first end 12 of the second tool 10b. In other words, the first end 12 of the second tool 10b is free of channels 20, 30, 32. Further, the first and second tools 10a, 10b may have different shapes and/or sizes. For example, the cross-sectional shape of the first tool 10a may be polygonal and the cross-sectional shape of the second tool 10b may be circular or rounded. Moreover, the perimeter of the first tool 10a may be equal to or smaller than the circumference 15 of the second tool 10b.

In embodiments, each of the first tool 10a, the second tool 10b and the plate 40 may be, individually or separately, sterilized and packaged prior to being provided to the user. The first and second tools 10a, 10b may be disposable tools capable of being disposed after the first and second tools 10a, 10b contour and shape the plate 40. The individually or separately sterilized and packaged first tool 10a, second tool 10b and plate 40 may be unpackaged before contouring and shaping the plate 40. After being unpackaged the fixed portions 45a, 45b of the sterile plate 40 may firmly and securely inserted into one of the channels 20, 30, 32 of the first and second sterile tools 10a, 10b. The sterile tools 10a, 10b may be moved in relationship to one another to apply deformation forces to the fixed portions 45a, 45b of the plate 40 firmly and securely held within the first and second tools 10a, 10b, respectively. The deformation forces applied by moving the first and second tools 10a, 10b deforms, bends and/or twists the deformation portion 48 of the plate 40 located between the first and second tools 10a, 10b. As a result, the plate 40 may be contoured by the deforming, bending and/or twisting of the deformation portion 48 of the plate 40.

It will be appreciated that various of the above-disclosed and other features and functions, or alternatives thereof, may be desirably combined into many other different systems and/or methods. Also, various presently unforeseen or unanticipated alternatives, modifications, variations or improvements therein may be subsequently made by those skilled in the art, and are also intended to be encompassed by the present disclosure.

We claim:

1. A contouring tool system for contouring a medical implant having a width, a height and a length defined between a first end of the medical implant and a second end of the medical implant located opposite to the first end of the medical implant, the system comprising:
a first tool having a length defined between a first end of the first tool and a second end of the first tool located opposite to the first end of the first tool, wherein the first tool comprising a first channel located adjacent to the first end of the first tool, wherein the first channel is a lateral channel extending across a width of the first tool, wherein the first channel has a first depth sized to receive the width of the medical implant when the medical implant is inserted into the first channel, wherein the first channel has a first width that is substantially the same as the height of the medical implant such that a first portion of the length of the medical implant is secured to the first tool when the medical implant is inserted into the first channel; and
a second tool having a length defined between a first end of the second tool and a second end of the second tool located opposite to the first end of the second tool, wherein a second channel is formed within the second tool, wherein the second channel has a second depth sized to receive the width of the medical implant when the medical implant is inserted into the second channel, wherein the second channel has a second width that is substantially the same as the height of the medical implant such that the second portion of the length of the medical implant is secure to the second tool when the medical implant is inserted into the second channel.

2. The contouring tool system according to claim 1, further comprising:
a third channel formed within the first tool, wherein the third channel is a longitudinal channel extending along at least a portion of the length of the first tool.

3. The contouring tool system according to claim 2, wherein the third channel extends an entirety of the length of the first tool from the first end of the first tool to the second end of the first tool.

4. The contouring tool system according to claim 2, further comprising:
a fourth channel formed within the first tool, wherein the fourth channel is lateral channel located adjacent to the second end of the first tool and extending across the width of the first tool.

5. The contouring tool system according to claim 1, wherein the first channel formed in the first tool is substantially perpendicular with respect to the length of the first tool.

6. The contouring tool system according to claim 5, wherein the first channel formed in the first tool is angled at an angle with respect to the length of the first tool, wherein the angle is less than ninety degrees.

7. The contouring tool system according to claim 1, further comprising:
a contouring guide surface extending from an outer circumference of the first tool to the first end of the first tool, wherein the surface has a cross-sectional profile that is curved.

8. The contouring tool system according to claim 7, wherein the contouring guide surface is a convex surface.

9. The contouring tool system according to claim 1, further comprising:
a third channel formed in the second tool extending across an entirety of the length of the second tool or across a width of the second tool and adjacent to the first end of the second tool.

10. A contouring tool system for contouring at least one medical implant, the system comprising:
a bone plate having a width, a height and a length defined between a first end of the bone plate and a second end of the bone plate located opposite to the first end of the bone plate;
a first tool having a length defined between a first end of the first tool and a second end of the first tool located opposite to the first end of the first tool, wherein the first tool comprising a first channel located adjacent to the first end of the first tool and extending across a width of the first tool, wherein the first channel has a first depth and a first width that are sized to receive and hold a first fixed portion of the bone plate; and
a second tool having a width and a length defined between a first end of the second tool and a second end of the second tool located opposite to the first end of the second tool, wherein a second channel is formed within the second tool and extends across the width of the second tool or an entirety of the length of the second tool, wherein the second channel has a second depth and a second width sized to receive and hold a second fixed portion of the bone plate,
wherein the first fixed portion of the bone plate is held within the first channel of the first tool and the second fixed portion of the bone plate is held within the second channel of the second tool after the bone plate is inserted into the first channel of the first tool and the second channel of the second tool.

11. The contouring tool system according to claim 10, wherein the depths of the first channel and the second channel are equal to or greater than the width of the bone plate.

12. The contouring tool system according to claim 10, wherein the widths of the first channel and the second channel are substantially equal to the height of the bone plate.

13. The contouring tool system according to claim 10, further comprising:
a third channel formed within the first tool extending along the length of the first tool or across the width of the first tool and adjacent to the second end of the first tool, wherein the third channel has a third depth that is equal to or greater than the width of the bone plate and a third width that is substantially equal to the height of the bone plate.

14. The contouring tool system according to claim 10, further comprising:
a contouring guide surface extending from an outer circumference to the first end of the first tool, wherein the contouring guide surface has a cross-sectional profile that is curved.

15. The contouring tool system according to claim 14, wherein the contouring guide surface is a convex surface.

16. A method for contouring a medical implant, the method comprising:
providing a first tool, a second tool and a bone plate, wherein each of the first tool, the second tool and the bone plate are individually sterilized and packaged;
unpackaging the first tool, the second tool and the bone plate;
inserting a first fixed portion of the bone plate into a first channel formed within the first tool, wherein the first channel extends along a first length of the first tool or across a first width of the first tool and adjacent to an end of the first tool, wherein the first channel is sized to hold the first fixed portion within the first tool;
inserting a second fixed portion of the bone plate into a second channel formed within the second tool, wherein the second channel extends along a second length of the second tool or across a second width of the second tool and adjacent to an end of the second tool, wherein the second channel is sized to hold the second fixed portion within the second tool; and
applying deformation forces to at least one portion of the bone plate by moving the first tool relative to the second tool, wherein the deformation forces cause a deformation within a deformation portion of the bone plate for contouring the bone plate, wherein the deformation portion of the bone plate is located between the first and second tools.

17. The method according to claim 16, wherein moving the first tool relative to the second tool comprises at least one of pushing, pulling, twisting, turning and bending the first tool with respect to the second tool for causing the deformation in the deformation portion of the bone plate.

18. The method according to claim 16, further comprising
pivoting the first tool with respect to the second tool via a fulcrum provided between the first tool and the second tool.

19. The method according to claim 18, wherein a contouring guide surface of the first tool is the fulcrum located between the first tool and the second tool, wherein the contouring guide surface of the first tool has a cross-sectional profile that is curved.

20. The method according to claim 19, wherein the contouring guide surface of the first tool is a convex surface.
